(19) United States
(12) Patent Application Publication
(10) Pub. No.: US 2009/0048500 A1
(43) Pub. Date: Feb. 19, 2009

(54) METHOD FOR USING A NON-INVASIVE CARDIAC AND RESPIRATORY MONITORING SYSTEM

(75) Inventor: Stephen Bruce Corn, Sharon, MA (US)

Correspondence Address: LAHIVE & COCKFIELD, LLP;FLOOR 30, SUITE 3000, ONE POST OFFICE SQUARE, BOSTON, MA, 02109, US

(73) Assignee: RESPIMETRIX, INC., Branford, CT (US)

(21) Appl. No.: 11/308,675

(22) Filed: Apr. 20, 2006

Related U.S. Application Data
(60) Provisional application No. 60/672,678, filed on Apr. 20, 2005.
(60) Provisional application No. 60/672,600, filed on Apr. 20, 2005.
(60) Provisional application No. 60/672,659, filed on Apr. 20, 2005.
(60) Provisional application No. 60/672,680, filed on Apr. 20, 2005.
(60) Provisional application No. 60/672,681, filed on Apr. 20, 2005.

(51) Int. Cl. A61B 5/1455

(52) U.S. Cl. 600/315

(57) ABSTRACT

A non-invasive monitoring system using radiated energy to identify cardiac and respiratory waveforms in patients is discussed. The monitoring system illuminates a subject in radiated energy and then detects the reflected radiated energy caused by respiratory and/or cardiac functions. The detected reflections are used to plot a two-dimensional waveform. The waveforms represent the rise and fall of a detected signal (the reflected energy) over time and are indicative of the small movements of the patient's chest and abdomen that are associated with cardiac and respiratory function. Different implementations of the monitoring system use laser or ultrasonic energy to capture breathing and cardiac waveforms for analysis. The waveforms may be used to diagnose the effectiveness of prescribed sleep medication, perform remote line of sight monitoring from a remote location, identify crying waveforms for babies and infants, identify cardiac waveforms in a non-invasive manner and identify seizure and tremor activity.

### CROSS REFERENCE TO RELATED APPLICATIONS

The present application is related to and claims the benefit of: U.S. Provisional Patent Application No. 60/672,678, filed Apr. 20, 2005, entitled “Medicine's First In-Home, Evidence-Based Medication Response System”, U.S. Provisional Patent Application No. 60/672,600, filed Apr. 20, 2005, entitled “Smart Infant Monitor and Effector ‘Watch Band’ Technology”, U.S. Provisional Patent Application No. 60/672,659, filed Apr. 20, 2005 entitled “Hand-Held Non-Contact Heart Rate and Respiratory Rate Monitor”, U.S. Provisional Patent Application No. 60/672,680 filed Apr. 20, 2005, entitled “Non-Contact Heart Rate and Rhythm Detection”, and U.S. Provisional Patent Application No. 60/672,681, filed Apr. 20, 2005 entitled “Neuro-Degenerative Monitoring System”.

### FIELD OF THE INVENTION

The illustrative embodiment of the present invention relates generally to non-invasive cardiac and respiratory monitoring systems and more particularly to the usage of non-invasive cardiac and respiratory monitoring systems

### BACKGROUND OF THE INVENTION

Conventional monitoring systems have been developed to monitor a number of medical conditions. For example, hospitals frequently use cardiac monitoring systems to generate a current view of a medical patient's current cardiac rate and rhythm. Similarly, respiratory monitoring systems are used to keep track of a patient's breathing rate. Conventional breathing and cardiac monitoring systems however are heavily reliant on sensors that are in physical contact with the patient being monitored. The reliance by conventional monitoring systems on sensors in physical contact with the patient presented a number of difficulties including the presence of wires (for non-wireless systems), the possibility of patient movement dislodging the sensors and patient discomfort from having the sensors physically attached.

In response to some of these concerns, non-invasive monitoring systems to allow the study of sleep apnea have been developed. The non-invasive monitoring systems use a beam of radiated energy such as laser and ultrasonic energy to illuminate a subject and capture the reflections of the energy caused by patient breathing to generate a waveform indicative of a breathing rate. Unfortunately, there are number of other areas that could also benefit from non-invasive respiratory and cardiac monitoring. For example, there are no well established home-based methods to objectively evaluate the response to a medication intended to affect respiration or breathing. It would be desirable to allow monitoring of babies during sleep that struck a proper balance between sensitivity and providing specific information. Non-conventional triage situations could also benefit from the use of a non-invasive respiratory monitoring system. Likewise, it would be beneficial to utilize non-contact analysis of heart rate and rhythm for clinicians and patients. Finally, a non-invasive monitoring system that was able to monitor seizure and tremor activity without relying on video means, direct observation or EEG telemetry would also be beneficial.

### BRIEF SUMMARY OF THE INVENTION

The illustrative embodiment of the present invention provides a method for drug efficacy monitoring, infant monitoring, remote respiratory monitoring, non-invasive cardiac monitoring and remote seizure disorder monitoring. The present invention utilizes a monitoring system using radiated energy to diagnose cardiac and respiratory rates in patients in a non-invasive manner. In one implementation, ultrasonic energy is used to capture breathing and cardiac waveforms for analysis.

In one aspect of the present invention a method of monitoring the efficacy of medication includes the step of injecting a subject with a selected medication. The method also includes the step of monitoring the subject with a non-invasive respiratory monitoring system. The monitoring system illuminates the subject with radiated energy produced from within a selected wavelength range, detects a reflection of the radiated energy from a surface that moves as a result of breathing by the subject, and identifies a waveform indicative of a breathing rate of the subject based on the reflection of the radiated energy. The method also stores the waveform indicative of the breathing rate and compares the stored waveform to a previously designated waveform to determine whether the selected medication caused an unacceptable deviation in the breathing pattern of the subject.

In another aspect of the present invention a method of monitoring the sleeping state of children includes the step of monitoring a subject with a non-invasive respiratory monitoring system. The monitoring system illuminates the subject with radiated energy produced from within a selected wavelength range, detects a reflection of the radiated energy from a surface that moves as a result of breathing by the subject, and identifies a breathing waveform indicative of the subject crying. The method also notifies programmatically a caregiver of the occurrence of the crying by the subject.

In an additional aspect of the present invention a method of remotely monitoring the respiratory rate of a subject includes the step of monitoring from a remote line of sight location a subject with a non-invasive portable respiratory monitoring system. The remote line of sight location is not physically adjacent to the subject. The respiratory monitoring system illuminates the subject with radiated energy produced from within a selected wavelength range, detects a reflection of the radiated energy from a surface that moves as a result of breathing by the subject, and identifies a waveform indicative of breathing rate of the subject based on the reflection of the radiated energy. The method also transmits the identified waveform to a remote location as part of a triage decision process.

In one aspect of the present invention a method of monitoring cardiac rate and cardiac rhythm includes the step of monitoring a subject with a non-invasive respiratory monitoring system. The monitoring system illuminates the subject with radiated energy produced from within a selected wavelength range, detects a reflection of the radiated energy from a surface that moves as a result of cardiac contractions by the subject, and identifies a waveform indicative of a cardiac rate and rhythm of the subject based on the reflection of the radiated energy. The method also compares the waveform indicative of the cardiac rate and rhythm to a previously supplied waveform in order to detect anomalies.

In another aspect of the present invention a method of monitoring seizure and tremor activity in a subject patient includes the step of monitoring a subject with a non-invasive monitoring system. The monitoring system illuminates the subject with radiated energy produced from within a selected wavelength range, detects a reflection of the radiated energy from a surface that moves as a result of at least one of breathing by the subject and cardiac contractions by the subject, and identifies a waveform indicative of either a breathing rate and/or a cardiac rate and rhythm of the subject based on the reflection of the radiated energy. The method also compares the waveform indicative of either a breathing rate and/or a cardiac rate and rhythm to a previously supplied waveform in order to identify seizure activity and/or tremor activity.

In one aspect of the present invention, a method of monitoring the efficacy of a treatment modality includes the step of monitoring a subject with a non-invasive respiratory monitoring system. The monitoring system illuminates the subject with radiated energy produced from within a selected wavelength range, detects a reflection of the radiated energy from a surface that moves as a result of breathing by the subject, and identifies an initial waveform indicative of a breathing rate of the subject based on the reflection of the radiated energy. The method also prescribes a treatment modality for the subject. Additionally the method includes the step of monitoring the subject subsequent to the application of the treatment modality with a non-invasive respiratory monitoring system. The monitoring system illuminates the subject with radiated energy produced from within a selected wavelength range, detects a reflection of the radiated energy from a surface that moves as a result of breathing by the subject, and identifies a second waveform indicative of a breathing rate of the subject based on the reflection of the radiated energy. The method then compares the initial waveform to the second waveform to determine whether the treatment modality resulted in a deviation in the breathing pattern of the subject.

### BRIEF DESCRIPTION OF THE DRAWINGS

The invention is pointed out with particularity in the appended claims. The advantages of the invention described above, as well as further advantages of the invention, may be better understood by reference to the following description taken in conjunction with the accompanying drawings, in which:

FIG. 1 depicts an environment suitable for practicing the illustrative embodiment of the present invention;

FIG. 2 is a flowchart of a sequence of steps followed by the illustrative embodiment of the present invention to monitor the efficacy of a medication;

FIG. 3 is a flowchart of a sequence of steps followed by the illustrative embodiment of the present invention to monitor the sleeping state of a baby;

FIG. 4 is a flowchart of a sequence of steps followed by the illustrative embodiment of the present invention to monitor a subject from a remote line of sight location;

FIG. 5 is a flowchart of a sequence of steps followed by the illustrative embodiment of the present invention to identify a cardiac waveform; and

FIG. 6 is a flowchart of a sequence of steps followed by the illustrative embodiment of the present invention to identify seizure and/or tremor activity.

### DETAILED DESCRIPTION OF THE INVENTION

The illustrative embodiment of the present invention utilizes a non-invasive monitoring system using radiated energy to identify cardiac and respiratory waveforms in patients. The monitoring system illuminates a subject in radiated energy and then detects the reflected radiated energy caused by respiratory and/or cardiac functions. The detected reflections are used to plot a two-dimensional waveform. The waveforms represent the rise and fall of a detected signal (the reflected energy) over time and are indicative of the small movements of the patient's chest and abdomen that are associated with cardiac and respiratory function. Different implementations of the monitoring system use laser or ultrasonic energy to capture breathing and cardiac waveforms for analysis. The waveforms may be used to diagnose the effectiveness of prescribed sleep medication, perform remote line of sight monitoring from a remote location, identify crying waveforms for babies and infants, identify cardiac waveforms in a non-invasive manner and identify seizure and tremor activity.

An exemplary monitoring system that may be used in the practice of the illustrative embodiment of the present invention was described in U.S. Pat. No. 6,062,216 (hereafter the '216 patent). The '216 patent (the contents of which are hereby incorporated by reference) describes a monitoring system using laser energy or ultrasonic energy to monitor respiratory function so as to detect sleep apnea. Although the '216 monitoring system was designed to monitor sleep apnea it may be leveraged to practice the present invention as discussed further below. Those skilled in the art will recognize that although the monitoring system of the '216 patent has been cited as an exemplary monitoring system which may be used in present invention, other non-invasive monitoring systems utilizing laser or ultrasonic energy to detect respiratory and/or cardiac waveforms may also be used within the scope of the present invention.

As noted above, the monitoring system of the present invention may use ultrasound to establish the waveforms used in the present invention. Ultrasonic sound is a vibration at a frequency above the range of human hearing, in other words usually in a range above 20 kHz. A shaped transducer in the monitoring system radiates a preferably continuous beam of ultrasound for example in the 25 kHz to 500 kHz range to illuminate a subject patient. Those skilled in the art will appreciate that other ranges may also be used without departing from the scope of the present invention. A receiving transducer in the monitoring system of the present invention or transducer array develops one or more signals which shift slightly from the incident frequency due to cardiac or respiratory motion. The signal is then analyzed and plotted to generate a waveform which may be compared against an appropriate benchmark. Appropriate adjustments are made by the monitoring system to account for the distance between the monitoring system and the subject as well as any environmental factors affecting the detection of the reflected energy.

In another implementation, the monitoring system may use laser detection means as described in the '216 patent in place of ultrasonic energy. In such a case a laser illuminates the subject patient in a beam of light of a selected wavelength and the reflected energy which varies based on respiratory and cardiac movements is traced so as to generate a waveform. Infrared or other wavelengths that are highly distinct from the spectral range of other light sources surrounding the subject may be selected so as to ease the detection of the reflected energy.

In a different implementation, the monitoring system may utilize ultra-wide band radar.

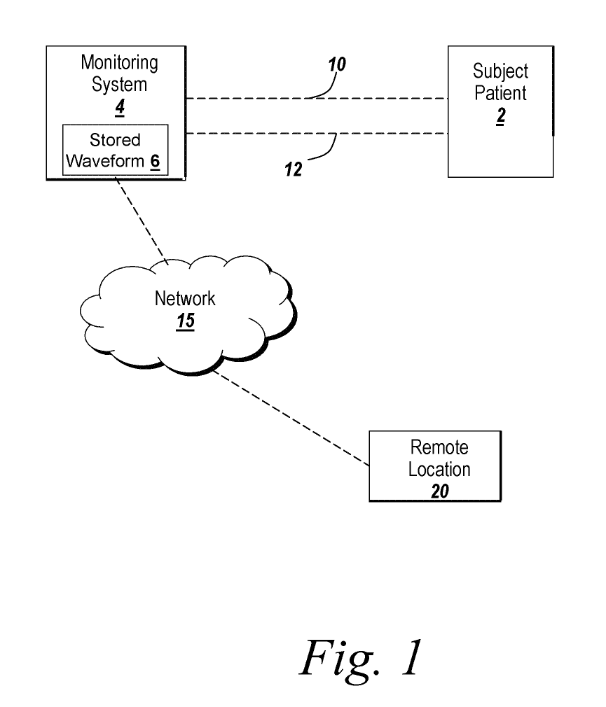

FIG. 1 depicts an environment suitable for practicing the illustrative embodiment of the present invention. A subject patient 2 is illuminated in a beam of energy 10 generated by the monitoring system 4 used in the present invention. The beam of energy 10 may be ultrasonic energy, laser energy or some other type of radiated energy enabling the monitoring system 4 to identify cardiac and respiratory waveforms. The monitoring system 4 detects reflections 12 in the radiated beam of energy 10 caused by the cardiac and/or respiratory functions of the patient subject 2. The detected reflections are used to generate waveforms identifying respiratory rates or cardiac rate and rhythm of the subject patient 2. In one implementation, the monitoring system 4 is also able to determine inspiratory/expiratory ratio (I:E ratio) from the acquired signal in order to provide additional clinical information on the effort of breathing for the subject patient 2. The monitoring system 4 may also determine “retraction” or uncoordinated movement of the chest and abdomen in the subject patient 2 based on the reflected energy. Similarly, the monitoring system 10 may be used to monitor obstructed breathing. It will be appreciated that the monitoring system 4 may analyze one or more detected reflections of radiated energy.

For example the monitoring system 4 may analyze an initial reflection and then further optimize the analysis with subsequent detected reflected signals. This would provide a benefit in situations where the subject is wearing bulky clothing.

The monitoring system 4 may include storage for one or more benchmark waveforms 6 which are used in comparisons with the identified cardiac and respiratory waveforms from the subject patient 2. The use of the benchmark waveforms 6 are discussed further below. In some implementations, the monitoring system 10 may be monitoring from a remote line of sight location that is not physically proximate to the subject patient 2 and may transmit the identified waveforms over a network 15 to a remote location 20 as part of distributed triage process. The use of the present invention to perform distributed triage is discussed further below.

As noted above, there are no well-established means to objectively evaluate the response to a medication or surgical procedures intended to affect respiration and/or breathing. For example, there is a growing search for pharmacologic agents to treat sleep disorders as the number of Americans alone is estimated as exceeding 82 million. However, medications for sleep problems are often prescribed without any objective means to monitor efficacy in the home setting. The illustrative embodiment of the present invention may be used to supply an objective means to measure an individual's response to specific drugs, dosages and combinations or treatment modalities, in a way that has never been achieved in the home setting. The monitoring system used in the present invention provides the ability to non-invasively and without contact, record, analyze and display the continuous breathing waveform of subjects both awake and asleep. The waveforms can be compared with benchmark waveforms to determine a patient's therapeutic response to drugs and monitor intended or unintended breathing effects. Amplitude, frequency, regularity, pauses, and periodicity can be accurately now studied with this technology. A patient being treated with a medicine intended to reduce apneic pauses can objectively learn if the medicine is having a positive impact on the breathing pattern. Drugs can be compared over time to see their impact on a patient's breathing. Side effects can be weighed against objective changes in breathing to determine the choice of drugs. Competitors can present objective data to show the benefit of one drug over another. Patients may be induced to switch from one drug brand to another based on objective data that the patient can gather in his or her sleep. Third party reimbursement companies may use this objective data in evaluating requests for more expensive drugs. Alternatively, patients may find the data generated by the monitoring system 10 extremely useful for reimbursement, since the data may objectively demonstrate a benefit of one drug over another. Additionally, the present invention may also be utilized to determine the effectiveness of a treatment modality by recording an initial waveform, having the patient undergo a treatment modality, and then recording a second waveform which may be compared with the initial waveform to determine the effectiveness of the treatment.

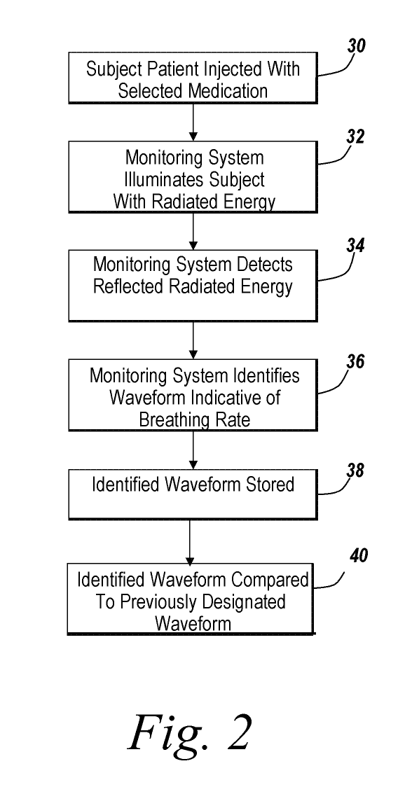

FIG. 2 is a flowchart of a sequence of steps followed by the illustrative embodiment of the present invention to monitor the efficacy of a medication. The sequence begins when a subject is injected with a selected medication (step 30). The monitoring system then illuminates the subject with radiated energy (step 32). The monitoring system detects reflections in the radiated energy that are caused by respiratory movements of the subject (step 34) and analyzes the detected reflections to identify waveforms indicative of a breathing rate of the subject (step 36). The identified waveform is stored (step 38) either on the monitoring system or in a separate storage location and the identified waveform is then compared to a benchmark waveform to determine the effect of the selected medication on the breathing rate of the subject (step 40). The comparisons may show a negligible effect, a desired effect or an excessive effect on the breathing rate.

The illustrative embodiment of the present invention may also be used to monitor the sleep state of an infant or small child. Baby monitors have been developed to allow parents and other caretakers to remotely assess the status of a child. That is, there are a variety of monitors that use microphone systems, either alone or in combination with a video system to help determine whether a child is awake, asleep or crying. One of the purposes of these remote monitors is to allow the caretakers to effectively assess the child, and maintain an excellent level of vigilance, while still allowing the caretakers to go on with their activities of daily living, or achieve a restful night sleep. Unfortunately, the current monitors do not provide a desirable balance between sensitivity and specificity. Additionally, the output of most of the monitors (microphone output of crying child) is just as disturbing as the actual event. Further, some of the monitors require direct contact or indirect contact (pad under the mattress) to accomplish the required sensing.

The present invention, employing ultrasonic or laser monitoring of a child's breathing, allows for the remote transmission of useful information about the child's breathing status to the child's caretakers. The monitoring system continuously and without the need for contact, monitors the child's breathing. In its simplest form, information can be remotely displayed showing the child's breathing pattern. Further, owing to the characteristic waveform of certain events such as “coughing”, “crying” and other breathing abnormalities, the monitoring system can alert the caretaker to the onset of coughing, crying or breathing abnormalities in a variety of ways. This graduated approach to notification of the caregiver is beneficial since it is known that babies intermittently arouse and cry during their naps and overnight sleep. Much of this arousal is self-limited, with the child returning to restful sleep in a matter of minutes without the need for caretaker intervention. Unlike a microphone system, the monitoring system in the present invention can be used to identify the onset of crying and then, present the data to the parent or caretaker in a user-friendly means, such as a flashing light on a screen signaling “crying” and/or a tone without actually having to listen to the crying. The signaling means (tone, visual, duration, delay, intensity, frequency) to the parents or caretakers can be customized to suite their needs. That is, the tone can be set to increase in volume and duration with the persistence of the crying. Alternatively, the monitoring system may detect the crying and monitor for its persistence. The caretaker may only be notified after the crying has continued for a preset time interval. This delayed notification feature would be of considerable value to all those parents and caretakers who previously would have been awakened from sleep with a loud cry via a microphone system, only to then have the child return to restful sleep.

The monitoring system may employ different notification methods. One such method is a “watch-band” alert system. In this implementation, with the recognition of crying by the monitoring system, information is sent to a watch-band worn by a caretaker and the caretaker is then alerted depending on the mode of activation chosen by the caretaker. For example, there should really be no need for both parents to be awakened every time a child cries by the transmitted sound to the parent's room. With this notification method, one parent can elect to wear the “watch-band” and be silently alerted to the crying by the triggering of a gentle increasing vibratory stimulus in the watch band. The notified parent can then tend to the child needs without ever waking the other parent or significant other. Similarly, multiple caretakers can wear “wrist watches” and the monitoring system could be set to alternate between the watch-bands in terms of alarming. For example, the alternating time interval could be set at two hours, so that each caretaker is assured that he or she will not be summoned for a two hour interval.

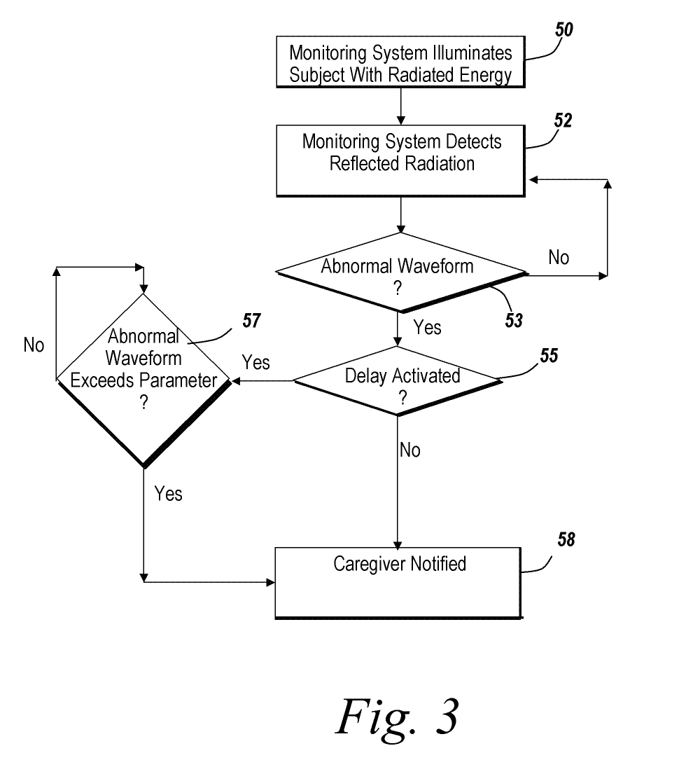

FIG. 3 is a flowchart of a sequence of steps followed by the illustrative embodiment of the present invention to monitor the sleeping state of a baby. The sequence begins as the monitoring system then illuminates the subject child with radiated energy (step 50). The monitoring system detects reflections in the radiated energy that are caused by respiratory movements of the subject (step 52) and analyzes the detected reflections to identify waveforms indicative of a breathing rate of the child. The monitoring continues until such time as an abnormal waveform indicative of coughing, crying or other breathing difficulty is detected (step 53). If a delay parameter is activated (step 55), the monitoring system compares the elapsed time from the onset of the detection of the abnormal waveform to a pre-selected time parameter (step 57). For example, some parents may wish to allow the child to cry for two minutes before being notified while others may wish the child to be allowed to cry for five minutes before notification. The monitoring system continues comparing the elapsed time to the time parameter until either the elapsed time exceeds the parameter, in which case notification of the caregiver is made (step 58), or the abnormal waveform is no longer detected. In the event the delay parameter has not been activated (step 55) the caregiver is immediately notified of the detection of the abnormal waveform (step 58).

In another implementation, the present invention may also be used to monitor the respiratory function of a subject from a distant line-of-sight location. For example, an emerging field in the military is physiological monitoring during combat and operations. A major focus of triaging of troops from helicopters and other “distant” line of sight locations is to accurately identify those who could benefit from assistance and those who could not. This triage decision-making is important for both the injured troops on the ground and for the safety of those who are making the rescue effort. For example, landing a helicopter to attempt resuscitation of an individual who cannot be resuscitated needlessly risks the rescuing team and aircraft, while diverting the rescuing team away from an injured member who may benefit from the arrival of the rescuing team. Similarly, every year, 25 million people are transported via ambulances to the emergency room. By the nature of their mission, ambulance teams frequently need to triage victims in non-conventional locations, often not immediately accessible to monitoring equipment. The use of the respiratory monitor of the present invention thus may serve as a triage tool by showing the breathing rate and pattern (normal, obstructive, apneic) of individuals even at great distances from the responding rescue team i.e., down a ravine or within a biologic contamination zone.

The illustrative embodiment of the present invention allows line of sight evaluation of an individual's respiration from a distance. A rescue team may determine from an airborne helicopter if an individual lying on the ground is undergoing normal respiration, obstructive breathing or no breathing at all. This information, including the breathing waveform, can also be remotely transmitted in real time to medics and other medical personnel behind the line of battle to make triage decisions in real time.

In one embodiment, an ultrasonic range finding module could be used as the basic sensing mechanism. This module acts much like a speaker/microphone pair. It emits sound waves at frequencies that are typically inaudible to humans. The sound waves propagate through the air, strike the target object and are partially reflected back towards the module. These reflected waves (echoes) can be detected by the module. Because sound waves travel through air at a known, fixed velocity, the distance to the target object can be calculated by measuring how much time elapses between when the wave was transmitted and when its echo is detected. Ultrasonic range finding systems are non-contact, safe and can be highly accurate. The monitoring system sends out sound waves and measures the distance to the target. By making these distance measurements frequently, such as at a rate of 100 times per second, the breathing rate of a subject may be determined.

The ultrasonic range finding module may include an ultrasonic transducer and appropriate detection and control circuitry. In one implementation, the module may emit narrow pulses of 220 kilohertz sound waves and detect return echoes that are generated when the sound waves strike a target. It will be appreciated that the module may emit pulses at other frequencies within the scope of the present invention. Because sound waves propagate in air at a rate of approximately 13044 in/second (at 0 degrees C.) the distance to the target object can be calculated by measuring the elapsed time between when the signal is transmitted and when its echo is detected. This corresponds to a delay of 0.9 milliseconds per foot of distance traveled. The ultrasonic range finding module is capable of very precise detection and time delay measurements and in one implementation has a resolution of 0.01. Furthermore the ultrasonic range finding module automatically compensates for errors that can be introduced by temperature variations (sound waves travel faster as the temperature increases). In one implementation, the ultrasonic range finding module is capable of performing measurements at a rate of 100 Hz, is highly directional and has an 8 degree conical beam. Motion artifact from the subject and/or user's location (helicopter, vehicle) will be damped by means commonly known in the art. For example, video camera motion suppression systems employing inertial filters in the x, y, and z planes easily allow for motion suppression.

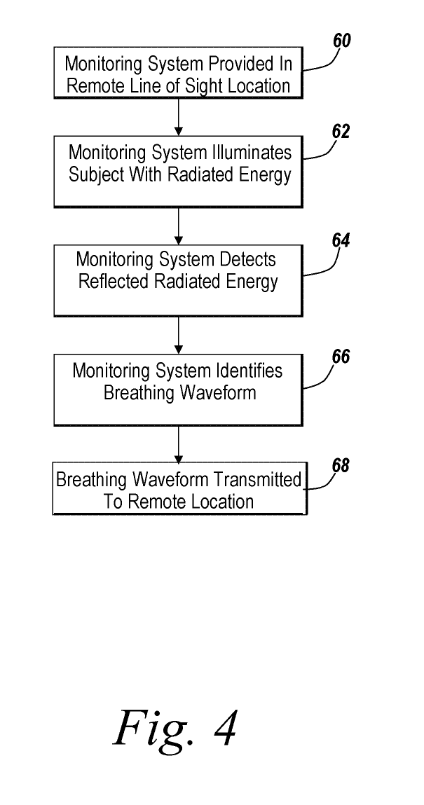

FIG. 4 is a flowchart of a sequence of steps followed by the illustrative embodiment of the present invention to monitor a subject from a remote line of sight location. The sequence begins by providing a monitoring system suitable for the present invention from a distant line of sight location not in physical proximity/not physically adjacent to the subject being monitored (step 60). Exemplary distant line of sight locations include airborne helicopters and vehicles. The monitoring system then illuminates the subject with radiated energy (step 62). The monitoring system detects reflections in the radiated energy that are caused by respiratory movements of the subject (step 64) and analyzes the detected reflections to identify waveforms indicative of a breathing rate of the subject (step 66). The waveforms may be analyzed at the distant line of site location. Alternatively, the identified waveform may be transmitted to a separate remote location for analysis as part of a distributed triage process (step 68).

In another aspect of the present invention, the monitoring system may be a handheld/portable monitoring system. There are approximately 3 million nurses and 800,000 physicians in the United States. These nurses and doctors need to stop to look at their watches for 30-60 seconds, multiple times per day for each and every patient in order to derive a respiratory rate for their patients. A handheld version of the monitoring system described above would allow quick respiration rates to be ascertained. Such a handheld monitoring system may be beneficial to EMTs firefighters, police, rescue and military personnel as well as hospital staff.

The illustrative embodiment of the present invention also allows a cardiac waveform showing a cardiac rate and/or rhythm to be identified. With each cardiac contraction, surface anatomy changes slightly. Pulse oximetry mechanisms use an algorithm for determining oxygen saturation that compensates for the small change in finger size with each pulse of blood. The monitoring system of the present invention uses either ultrasonic or laser signal emissions as discussed above to identify the change in body surface contour and distance from the monitoring system. Measuring this change in contour or distance with respect to time results in a waveform where the heart rate and/or rhythm are measurable.

Owing to the differences in heart rate and breathing rate intrinsic rates and amplitudes that are determined from surface anatomy, heart rate determinations can be made in breathing subjects. This is true even when the heart rate is measured over areas that move with respiration, such as the chest and abdomen. The heart rate is seen as smaller amplitude oscillations within the “slower” waveform of the respiratory rate.

In one embodiment an ultrasonic probe is used to measure the change in distance with respect to time of the surface of the body. The probe is aimed at the chest, abdomen, radial artery at the level of the wrist or the carotid artery at the level of the neck. Each approximately sinusoidal waveform measured would demonstrate the heart rate (waves per minute) and rhythm (regular, irregular, regularly irregular). Amplitude of the waveform may also demonstrate useful information, such as the volume status of the patient, strength of the pulse and/or contractile force.

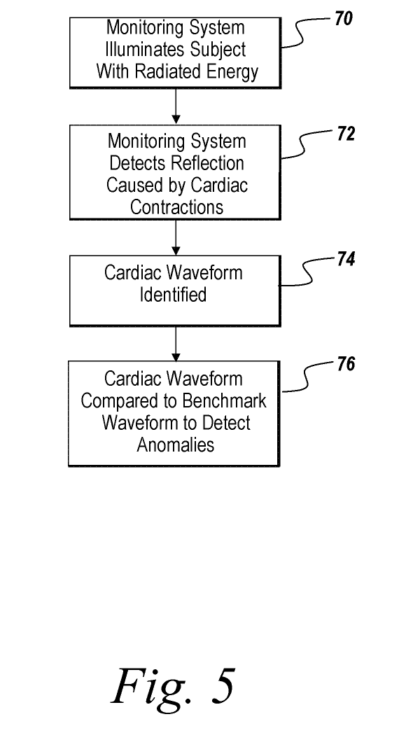

FIG. 5 is a flowchart of a sequence of steps followed by the illustrative embodiment of the present invention to identify a cardiac waveform. The monitoring system illuminates a subject with radiated energy (step 70). The monitoring system then detects reflections in the radiated energy that are caused by cardiac related contractions of the subject (step 72) and analyzes the detected reflections to identify waveforms indicative of a cardiac rate and/or rhythm of the subject (step 74). The identified cardiac waveform may then be compared to a benchmark cardiac waveform to detect anomalies present in the subject's cardiac function.

In an additional aspect of the present invention, the monitoring system may be used to detect seizure disorders. Seizure disorders are a common neurological problem. In the United States alone, it has been estimated that more than 4 million people have some form of epilepsy. The prevalence has been estimated to be about 5 to 8 in every 1,000 people and 200,000 new cases are diagnosed each year. Seizures are an episodic disorder of neuronal function that results in disturbances of sensation, motor function, thought and consciousness. Recurrent seizures or prolonged seizures can cause permanent injury to the brain. Seizures that last longer than 20 to 30 minutes can damage the brain's neurons. Patients suffering from seizure disorders are currently monitored by video, EEG telemetry, or direct observation.

The illustrative embodiment of the present invention provides a non-invasive monitoring option that may be used detect the onset of seizure activity. As noted above, the monitoring system of the present invention continuously measures the change in distance of the subject with respect to the probe by employing either ultrasound or laser distance determination methods. A waveform is generated which depicts normal rhythmic respiratory and heart rate surface body changes and gross motor changes. Both seizure and tremor activity have characteristic surface motion changes and gross motor movement changes. As such seizure and tremor activity is easily discerned from the waveform.

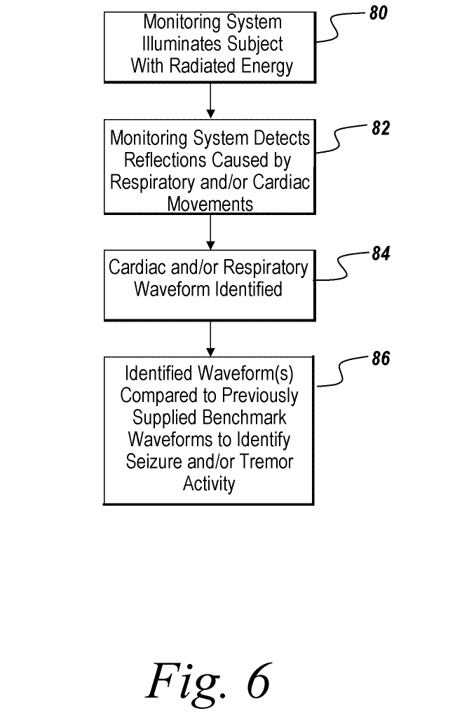

FIG. 6 is a flowchart of a sequence of steps followed by the illustrative embodiment of the present invention to identify seizure and/or tremor activity. The monitoring system illuminates a subject with radiated energy (step 80). The monitoring system then detects reflections in the radiated energy that are caused by cardiac related contractions and/or respiratory movements of the subject (step 82). The monitoring system analyzes the detected reflections to identify waveforms indicative of a cardiac rate and rhythm of the subject and/or a respiratory rate of the subject (step 84). The identified waveform may then be compared to a benchmark waveform to identify seizure or tremor activity.

In another aspect of the present invention, the respiratory monitor may be used to give bio-feedback to a subject. For example, during the course of the day, a subject may periodically sit down in front of a computing device or other device equipped with the monitoring system of the present invention in order to have a breathing and/or cardiac waveform identified in a non-invasive manner. In one implementation, the monitoring system may be built into a piece of exercise equipment being utilized by the subject.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

What is claimed as new and desired to be protected by Letters Patent of the United States is:

1. A method of monitoring the efficacy of medication, comprising:
providing a subject with a selected medication;
monitoring the subject with a non-invasive respiratory monitoring system, the monitoring system illuminating the subject with radiated energy produced from within a selected wavelength range, detecting a reflection of the radiated energy from a surface that moves as a result of breathing by the subject, and identifying a waveform indicative of a breathing rate of the subject based on the reflection of the radiated energy;
storing the waveform indicative of the breathing rate; and
comparing the stored waveform to a previously designated waveform to determine whether the selected medication caused an unacceptable deviation in the breathing pattern of the subject.

2. The method of claim 1 wherein the radiated energy is an ultrasonic transmission.

3. The method of claim 1 wherein the radiated energy is a laser transmission.

4. The method of claim 1 wherein the selected medication is designed to treat one of insomnia, apneic pauses and breathing abnormalities.

5. The method of claim 1 wherein the monitoring takes place in one of the home of the subject and a non-institutional setting.

6. A method of monitoring the sleeping state of children, comprising:
monitoring a subject with a non-invasive respiratory monitoring system, the monitoring system illuminating the subject with radiated energy produced from within a selected wavelength range, detecting a reflection of the radiated energy from a surface that moves as a result of breathing by the subject, and identifying a breathing waveform indicative of one of abnormal breathing by the subject, the subject crying and the subject coughing; and
notifying programmatically a caregiver of the occurrence of one of the subject abnormally breathing, coughing and crying.

7. The method of claim 6, further comprising:
tracking an elapsed time from the onset of the identification of the breathing waveform indicative of the subject crying;
comparing the elapsed time to a pre-determined parameter; and
performing said notifying only after the elapsed time exceeds said pre-determined parameter.

8. The method of claim 6 wherein the radiated energy is an ultrasonic transmission.

9. The method of claim 6 wherein the radiated energy is a laser transmission.

10. The method of claim 6 wherein said notifying further comprises:
providing an initial audible notification to the caregiver; and
incrementally increasing the level of the audible notification based upon the amount of time elapsed following the initial audible notification.

11. The method of claim 6 wherein said notifying further comprises:
providing a non-audible notification to a caregiver.

12. The method of claim 11 wherein the non-audible notification is delivered by programmatically initiating a vibrating motion of an accessory worn by the caregiver.

13. The method of claim 12, further comprising:
providing a plurality of caregivers each wearing an accessory capable of vibration; and
alternating the notification between the plurality of caregivers based upon a previously determined criteria.

14. A method of remotely monitoring the respiratory rate of a subject, comprising:
monitoring from a remote line of sight location a subject with a non-invasive portable respiratory monitoring system, the remote line of sight location not physically adjacent to the subject, the monitoring system illuminating the subject with radiated energy produced from within a selected wavelength range, detecting a reflection of the radiated energy from a surface that moves as a result of breathing by the subject, and identifying a waveform indicative of breathing rate of the subject based on the reflection of the radiated energy; and
transmitting the identified waveform to a remote location as part of a triage decision process.

15. The method of claim 14 wherein the radiated energy is an ultrasonic transmission.

16. The method of claim 14 wherein the radiated energy is a laser transmission.

17. A method of monitoring cardiac rate and cardiac rhythm, comprising:
monitoring a subject with a non-invasive monitoring system, the monitoring system illuminating the subject with radiated energy produced from within a selected wavelength range, detecting a reflection of the radiated energy from a surface that moves as a result of cardiac contractions by the subject, and identifying a waveform indicative of at least one of a cardiac rate and rhythm of the subject based on the reflection of the radiated energy; and
comparing the waveform indicative of the at least one of a cardiac rate and rhythm to a previously supplied waveform in order to detect anomalies.

18. The method of claim 17 wherein the radiated energy is an ultrasonic transmission.

19. The method of claim 17 wherein the radiated energy is a laser transmission.

20. The method of claim 17 wherein the waveform is used to determine a volume status of the subject.

21. The method of claim 17 wherein the waveform is used to determine a strength of a pulse of the subject.

22. The method of claim 17 wherein the waveform is used to determine a force of a cardiac contraction of the subject.

23. A method of monitoring seizure and tremor activity in a subject patient, comprising:
monitoring a subject with a non-invasive monitoring system, the monitoring system illuminating the subject with radiated energy produced from within a selected wavelength range, detecting a reflection of the radiated energy from a surface that moves as a result of at least one of breathing by the subject and cardiac contractions by the subject, and identifying a waveform indicative of at least one of a breathing rate and a cardiac rate and rhythm of the subject based on the reflection of the radiated energy; and
comparing the waveform indicative of the at least one of a breathing rate and a cardiac rate and rhythm to a previously supplied waveform in order to identify at least one of seizure activity and tremor activity.

24. The method of claim 23 wherein the radiated energy is an ultrasonic transmission.

25. The method of claim 23 wherein the radiated energy is a laser transmission.

26. The method of claim 23 wherein the waveform is used to identify at least one of surface body changes of the subject and gross motor changes of the subject.

27. A method of monitoring the efficacy of a treatment modality, comprising:
monitoring a subject with a non-invasive respiratory monitoring system, the monitoring system illuminating the subject with radiated energy produced from within a selected wavelength range, detecting a reflection of the radiated energy from a surface that moves as a result of breathing by the subject, and identifying an initial waveform indicative of a breathing rate of the subject based on the reflection of the radiated energy;
prescribing a treatment modality for the subject;
monitoring the subject subsequent to the application of the treatment modality with a non-invasive respiratory monitoring system, the monitoring system illuminating the subject with radiated energy produced from within a selected wavelength range, detecting a reflection of the radiated energy from a surface that moves as a result of breathing by the subject, and identifying an second waveform indicative of a breathing rate of the subject based on the reflection of the radiated energy; and
comparing the initial waveform to the second waveform to determine whether the treatment modality resulted in a deviation in the breathing pattern of the subject.
